(19) United States
(12) Patent Application Publication
(10) Pub. No.: US 2010/0292581 A1
(43) Pub. Date: Nov. 18, 2010

(54) Dynamic Calibration of an Optical Spectrometer

(76) Inventors: Peter Guy Howard, Junction City, OR (US); Steve C. White, LaQuinta, CA (US)

Correspondence Address: CHRISTIE, PARKER & HALE, LLP, PO BOX 7068, PASADENA, CA, 91109-7068, US

(21) Appl. No.: 12/779,926

(22) Filed: May 13, 2010

Related U.S. Application Data
(60) Provisional application No. 61/178,002, filed on May 13, 2009.

(51) Int. Cl. A61B 6/00; G01J 3/28

(52) U.S. Cl. 600/476; 356/326

(57) ABSTRACT

A dynamically calibrating optical spectrometer and method for dynamically calibrating the optical spectrometer are provided. The optical spectrometer may include a band-pass device and a device that scans, such that a scan window can be widened, and the edges of the band-pass device can be used to calibrate out drifts in the system. The optical spectrometer may be for acquiring and analyzing a spectroscopic sample of an analyte from a sampling region of the tissue of a person. The optical spectrometer includes a tunable source of electromagnetic radiation, a probe for delivering the electromagnetic radiation to the tissue at the sampling region and obtaining a diffuse reflectance signal from the tissue at the sampling region, a spectroscopic detector for analyzing the diffuse reflectance signal for presence of the analyte, and a wavelength calibration detector for calibrating the tunable source of electromagnetic radiation to a desired wavelength.

### CROSS-REFERENCE TO RELATED APPLICATION(S)

This application claims priority to and the benefit of U.S. Provisional Patent Application No. 61/178,002, entitled DYNAMIC CALIBRATION OF AN OPTICAL SPECTROMETER, filed May 13, 2009, the entire content of which is herein incorporated by reference. This application also contains subject matter related to the subject matter contained in U.S. patent application Ser. No. 12/498,360, entitled SYSTEM AND METHOD FOR NON-INVASIVE SPECTROSCOPIC DETECTION FOR BLOOD ALCOHOL CONCENTRATION, filed Jul. 6, 2009, and which claims priority to and the benefit of U.S. Provisional Patent Application No. 61/133,892, entitled SYSTEM FOR NON-INVASIVE SPECTROSCOPIC DETECTION OF BLOOD ALCOHOL CONCENTRATION, filed Jul. 3, 2008, the entire contents of which are herein incorporated by reference. This application also contains subject matter that is related to the subject matter contained in U.S. patent application Ser. No. 11/945,992, entitled APPARATUS FOR NON-INVASIVE SPECTROSCOPIC MEASUREMENT OF ANALYTES, AND METHOD OF USING THE SAME, filed Nov. 27, 2007, which claims priority to and the benefit of U.S. Provisional Patent Application Nos. 60/949,836, entitled APPARATUS AND METHOD FOR NON-INVASIVE SPECTROSCOPIC MEASUREMENT OF BLOOD ALCOHOL CONCENTRATION, filed Jul. 13, 2007, and 60/966,028, entitled APPARATUS AND METHOD FOR NON-INVASIVE SPECTROSCOPIC MEASUREMENT OF BLOOD ALCOHOL CONCENTRATION, filed Aug. 24, 2007, the entire contents of which are herein incorporated by reference. This application also contains subject matter that is related to the subject matter contained in U.S. patent application Ser. No. 11/702,806, entitled METHOD AND SYSTEM FOR PREVENTING UNAUTHORIZED USE OF A VEHICLE BY AN OPERATOR OF THE VEHICLE, filed Feb. 5, 2007, the entire content of which is herein incorporated by reference.

### BACKGROUND

The present invention relates generally to optical spectroscopy. More particularly, the present invention relates to the non-invasive detection of one or more substances or analytes such as the concentration of alcohol in human blood using optical spectroscopy. Specifically, various embodiments of the present invention provide for dynamic calibration of an apparatus for optical spectroscopy generally and in an exemplary embodiment to non-invasive detection of the blood alcohol concentration in a person using optical spectroscopy, which may also employ a biometric verification to provide both a detected blood alcohol concentration and a biometric identification to assure that the detected blood alcohol concentration is obtained from an identified person.

One problem long extant in the prior art is to provide a reliable device for non-invasive detection of a blood alcohol concentration in a person. Another problem is to provide for the non-invasive detection of the blood alcohol concentration in the person in a fast and repeatable manner. A further problem is to provide verification of the person subjected to the blood alcohol concentration analysis.

As disclosed in U.S. patent application Ser. No. 12/498,360, an optical spectrometer can be used to provide for the non-invasive detection of the blood alcohol concentration in a person. The optical spectrometer operates over a range of temperatures. The operation of various components of the optical spectrometer is affected by changes in temperature (thermal drift). Accordingly, the precision of the determination of the blood alcohol is also affected by changes in temperature.

Generally, most instrumentation is not operated in a temperature-controlled environment. The problem relating to optical spectrometers in general and, in particular, the optical spectrometer used to provide for the non-invasive detection of the blood alcohol concentration in a person, is that in at least two aspects they are not thermally stable.

In a specific sense, if the temperature changes in an exemplary optical spectrometer employing a piezoelectric element comprising a piezoelectrically actuated scanning Fabry-Perot etalon, the piezoelectric element will change in size due to the temperature change faster than quartz elements comprising the piezo etalon. Thus, the optical spectrometer will change its apparent tuning point as the air gap between the quartz elements controlled by the piezoelectric element changes with temperature.

In a more general sense, even if the piezoelectric element was not present and the quartz elements were static etalons all constructed from quartz, a large body problem exists. This is not much of a problem if the temperature changes relatively slowly, for the quartz elements achieve an insubstantial temperature gradient, the outside grows similarly to the inside, and they substantially compensate for themselves. Nevertheless, if the temperature changes at a rate faster than heat can migrate into the mass of the quartz elements, then they will be unstable until they can achieve their molecular equilibrium. Exemplary temperature changes include temperature fluctuations with the ambient temperature in the room and the heat produced from the optical spectrometer device during routine operation, especially when the spectrometer is initiated from a cold state.

Various embodiments of the present invention provide a solution for the reliable, fast, and repeatable operation of an optical spectrometer and thus, detection of the blood alcohol or other analyte concentration in an identified individual with improved precision. Advantageously, the various embodiments of the present invention provide dynamic calibration of the optical spectrometer to overcome the problem of thermal drift.

### SUMMARY

In accordance with the present invention, dynamic calibration of an optical spectrometer is provided for precise non-invasive spectroscopic detection, for example, of a substance or analyte present in the blood of a person, such as alcohol, its metabolic byproducts, or some other analyte, possibly in conjunction with a biometric verification of that person. The dynamic calibration provided by the embodiments of the present invention may advantageously be incorporated in a portable device, such as a hand-held device, which may be powered using a portable power supply, such as a battery.

In accordance with the example application of detection of blood alcohol concentration, the spectroscopic detection may be accomplished by impinging electromagnetic radiation on the tissue of a person, then acquiring and analyzing electromagnetic radiation resulting from the interaction with the tissue of the person. For example, the spectroscopic detection may be accomplished by way of an articulated probe head that applies a consistent pressure and angle to an interstitial region between the index and middle fingers above the web of a person whose blood alcohol concentration is being detected coupled to the optical spectrometer. The probe head incorporates a fiber optic bundle that transmits and receives electromagnetic radiation impinged on the interstitial region to perform spectroscopic detection by the optical spectrometer, which is dynamically calibrated to provide improved precision of the spectrometer.

In accordance with another aspect of the present invention, the spectroscopic detection of the blood alcohol concentration in a person is performed in conjunction with biometric verification of that person for authentication of the detection that is performed. The biometric verification may be accomplished by way of a fingerprint scan, for example. In accordance with one aspect of the present invention, the dynamic calibration can be cross-correlated using data obtained at different times on the same identified person, further improving precision.

According to an exemplary embodiment of the present invention, a method of dynamic calibration of an optical spectrometer comprising a spectroscopic detector for analyzing a spectroscopic sample of an analyte obtained through directing a tunable source of electromagnetic radiation at the tissue of a person through a probe is provided. The method includes directing a portion of the electromagnetic radiation to a wavelength calibration detector, measuring the electromagnetic radiation at the wavelength calibration detector, and tuning the source of electromagnetic radiation using the wavelength calibration detector measurements to correspond to a desired wavelength of the electromagnetic radiation.

The optical spectrometer may further include a diffuse reflectance surface for calibrating the spectroscopic detector. The method may further include directing the source of electromagnetic radiation to the diffuse reflectance surface to provide a spectroscopic baseline sample for the spectroscopic detector.

The optical spectrometer may further include a shutter or door for occluding the probe. The shutter or door includes the diffuse reflectance surface. The method may further include occluding the probe with the shutter or door, and acquiring the spectroscopic baseline sample.

The shutter or door may be further for automatically and temporarily occluding the probe. The method may further include automatically and temporarily occluding the probe for obtaining the spectroscopic baseline sample at a time proximal to obtaining the spectroscopic sample of the person.

The optical spectrometer may further include a band-pass filter for filtering the electromagnetic radiation to a desired wavelength range. The method may further include acquiring a plurality of spectroscopic baseline samples of the diffuse reflectance surface, and tuning the source of electromagnetic radiation using the baseline samples to deliver the electromagnetic radiation at a plurality of desired wavelengths within the desired wavelength range.

According to another exemplary embodiment of the present invention, a dynamically calibrating optical spectrometer for acquiring and analyzing a spectroscopic sample of an analyte from a sampling region of the tissue of a person is provided. The optical spectrometer includes a tunable source of electromagnetic radiation, a probe for delivering the electromagnetic radiation to the tissue at the sampling region and obtaining a diffuse reflectance signal from the tissue at the sampling region, a spectroscopic detector for analyzing the diffuse reflectance signal for presence of the analyte, and a wavelength calibration detector for calibrating the tunable source of electromagnetic radiation to a desired wavelength.

The optical spectrometer may further include a diffuse reflectance surface for providing a spectroscopic baseline sample for calibrating the spectroscopic detector.

The diffuse reflectance surface may be configured to temporarily occlude the probe for acquiring the spectroscopic baseline sample.

The diffuse reflectance surface may be configured to automatically occlude the probe, for acquiring the spectroscopic baseline sample at a time proximal to acquiring the spectroscopic sample of the person.

The optical spectrometer may further include a door or shutter including the diffuse reflectance surface.

The diffuse reflectance surface may include Spectralon.

The optical spectrometer may further include a band-pass filter for filtering the electromagnetic radiation to a desired wavelength range. The optical spectrometer may be further for using a plurality of spectroscopic baseline samples of the diffuse reflectance surface to tune the source of electromagnetic radiation to deliver the electromagnetic radiation at a plurality of desired wavelengths within the desired wavelength range.

The analyte may be alcohol.

The optical spectrometer may further include a biometric sensor for verifying the identity of the person.

The biometric sensor may be a fingerprint scanner.

The optical spectrometer may further include a calibration arm for directing a portion of the electromagnetic radiation from the tunable source to the wavelength calibration detector.

The calibration arm may include a fixed etalon.

The optical spectrometer may further include an adjustable etalon for tuning the source of the electromagnetic radiation to the desired wavelength.

According to yet another exemplary embodiment of the present invention, a dynamically calibrating optical spectrometer for acquiring and analyzing a spectroscopic sample of an analyte from a sampling region of the tissue of a person is provided. The optical spectrometer includes a tunable source of electromagnetic radiation, a probe for delivering the electromagnetic radiation to the tissue at the sampling region and obtaining a diffuse reflectance signal from the tissue at the sampling region, a spectroscopic detector for analyzing the diffuse reflectance signal for presence of the analyte, and a diffuse reflectance surface for providing a spectroscopic baseline sample for calibrating the spectroscopic detector.

The diffuse reflectance surface may be configured to automatically occlude the probe, for acquiring the spectroscopic baseline sample at a time proximal to acquiring the spectroscopic sample of the person.

The foregoing and other objects, features, and advantages of the present invention will become more readily apparent from the following detailed description of various embodiments of the present invention, which proceeds with reference to the accompanying drawings.

### BRIEF DESCRIPTION OF THE DRAWINGS

The various embodiments of the present invention will be described in conjunction with the accompanying drawings to facilitate an understanding of the present invention. In the drawings, like reference numerals refer to like elements. In the drawings:

FIG. 1 is a diagram of an exemplary hand cradle housing that contains a probe head, probe base, and biometric sensor according to an embodiment.

FIG. 2 is a front view of the probe head and probe base of the housing of FIG. 1. Included in the probe head are the optical elements or other device for transmitting and receiving electromagnetic radiation used to perform spectroscopic detection. The probe base may house the optics for the probe head and provide support for the probe head.

FIG. 3 is a schematic diagram of the optical portion of an optical spectrometer that undergoes dynamic calibration in accordance with one embodiment of the present invention.

FIG. 4 is an architectural block diagram of the optical spectrometer that undergoes dynamic calibration in accordance with one embodiment of the present invention.

FIG. 5, comprising FIGS. 5A and 5B, illustrates spectra resulting from spectroscopic analysis of the tissue of a person for the detection of blood alcohol concentration.

FIG. 6 is a schematic drawing of the piezo-electrically actuated scanning Fabry-Perot etalon of the optical spectrometer shown in FIG. 3.

FIG. 7, comprising FIGS. 7A and 7B, illustrates the optical spectrum produced by the piezo etalon shown in FIG. 6 and a corresponding staircase voltage curve representing the voltages applied to the piezoelectric element.

### DETAILED DESCRIPTION

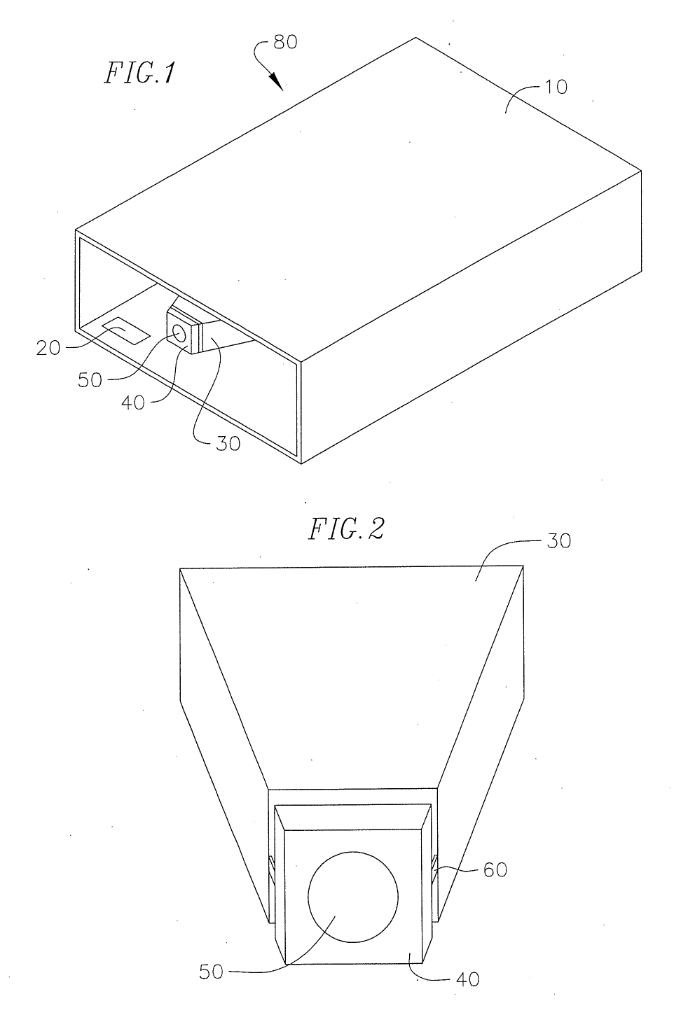

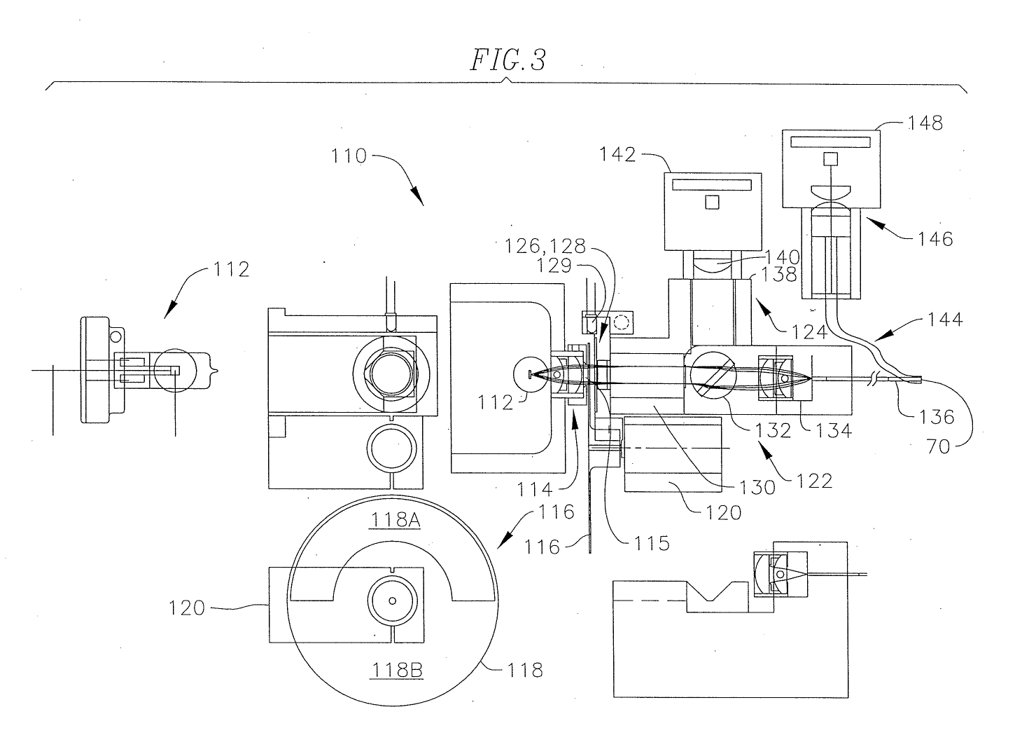

FIGS. 1 and 2 illustrate an exemplary hand cradle 80 of an optical spectrometer that is dynamically calibrated in accordance with the present invention. A probe head 40 is mounted to a probe base 30. The probe head 40 may be positioned to obtain unique information to identify a person in conjunction with detection of the blood alcohol concentration of the person. Verification of the person may be determined before or contemporaneously with the detection procedure that is performed at a predetermined sampling region of the person. The probe head 40 is configured within a housing 10 to assure that spectroscopic detection of the person is performed reliably.

The sampling region may be the interstitial region between fingers, for example the interstitial region between the index and middle fingers above the web of a hand of the person. The sampling region may also be between the toes of the person. The region adjacent to a single finger or toe may also serve as the sampling region. The size of the housing may accommodate a single finger or toe, two fingers or toes, multiple fingers or toes, or the entire hand or foot of a person taking into account variations of physical size of extremities within the general population. The housing 10 may be sized to accommodate any two consecutive fingers of the hand of a person and, more particularly, the index and middle fingers of a person.

The probe head 40 may be mounted to the probe base 30 by way of a flexible hinge 60, which enables rotation of the probe head in a vertical plane. Rotation of the probe head 40 assures that a fiber optic bundle 50 incorporated into the probe head 40 seats flush against the tissue in an interstitial region between the fingers of a person, which may serve as a sampling region. The probe head 40 and probe base 30 combine to provide the probe head 40 rotational and translational freedom while mounted in the housing 10. The rotational freedom of the probe head 40 enables the probe head to conform to the contour of the tissue of the person in the sampling region by varying the angle of the probe head 40 with respect to the tissue of the person. The translational freedom of the probe head 40 enables the probe head to impart a consistent pressure on the tissue of the person in the sampling region.

The probe head 40 acquires readings in the interstitial region between the fingers, for example, where there is lower muscle density such as between the palmar interossei and dorsal interossei muscles. Other portions of the finger that are low in muscle density may be appropriate as sampling regions as well. Detecting between the muscles provides results that are more representative of substances or analytes in the blood, with less interference from variations due to constituents in the muscle, such as lactic acid that may produce less reliable results in the detection of the blood alcohol concentration. The interstitial region between adjacent fingers of a person is minimally affected by the presence of lactic acid in muscle tissue.

In operation, a person inserts two fingers into the housing 10, which contains the probe head 40 and probe base 30. The housing 10 is large enough to accommodate fingers of various sizes to account for variations within the general population, yet is small enough to prevent tampering with the probe head 40 or a biometric sensor 20. The biometric sensor 20 may be mounted near the aperture of the housing 10. In this manner, biometric verification establishing the identity of the person can be accomplished before the spectroscopic detection is performed, and the spectroscopic detection cannot be performed without the biometric authentication of the same person whose blood alcohol concentration is detected.

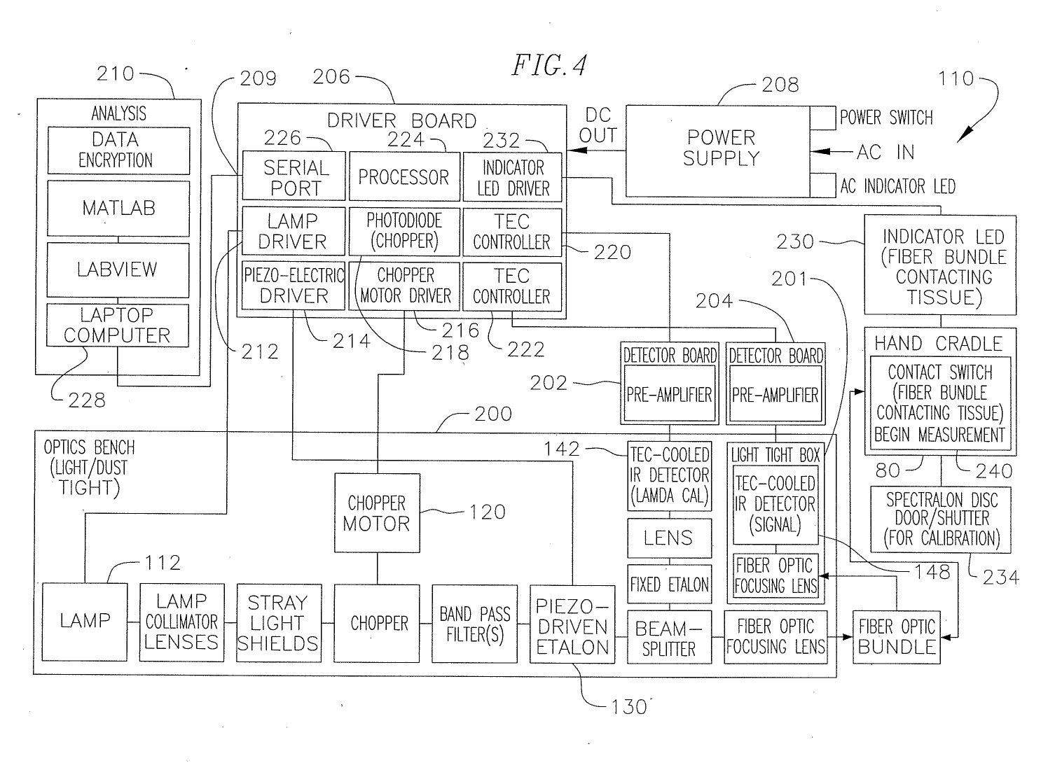

FIG. 3 is a schematic diagram of an optical spectrometer 110 that is dynamically calibrated in accordance with one embodiment of the present invention. The optical spectrometer 110 comprises a lamp 112 that provides a source of light. The light produced by the lamp 112 is then collimated by collimation lenses 114, which may be constructed from calcium fluoride. The light passing through the collimation lenses 114 is radiated through an aperture 115 to prevent light that is not collimated from being transmitted.

The spectroscopic detector 110 also comprises a chopper device 116 on which collimated light impinges. The chopper device 116 comprises a chopper wheel 118 driven by a chopper motor 120. The chopper wheel 118 has an arcuate slot 118A, which transmits light, and an opaque portion 118B, which masks light, so collimated light is either on or off depending upon the rotation of the chopper wheel 118. In one implementation, the chopper motor 120 rotates the chopper wheel 118 at 10,000 rpm. The principle of operation of the chopper device 116 is well understood by persons skilled in the art.

In another embodiment, an LED may be employed as the light source. The advantage of an LED is that the LED may be configured as a pulsed light source, which eliminates the need for a chopper having the spinning chopper wheel 118 to reduce the number of moving parts. As will be described in more detail below, the light from the light source is pulsed, for example, by the chopper device 116, or by a pulsed LED.

During the period that the light is off, dark current is integrated. During the period that the light is on, the amplitude of light received by a detector is detected by integrating the dark current signal produced by the detector and comparing the signal produced when light is received by an alcohol signal detector.

The chopper device 116 not only feeds light from the light source, but also provides a feedback timing signal to the optical spectrometer. That is, the chopper device 116 both supplies pulsed light, as well as provides a timing signal that the spectrometer uses to be sure it is integrating the dark current at the correct point in the chopper wheel's rotation. The chopper device 116 is employed because the dark current is relatively high compared to the light sensed by the alcohol signal detector.

One implementation is shown to assure that a sufficient number of photons is impinged on the tissue of the person whose blood alcohol concentration is being detected. Accordingly, FIG. 3 shows a sampling-region illuminating arm 122 and a wavelength calibration arm 124.

As shown in FIG. 3, the light passing through the chopper device 116 is impinged on light band-pass filters 126 and 128. The reason for two band-pass filters is that the lamp 112 is employed instead of an LED, so there is a great deal more light in the visible portion of the spectrum. Because the band-pass filter 126, for example, has to be narrow and have an optimum pass characteristic over the operating region of the spectrum, there is a tendency that the band-pass filter 126 may transmit light in shorter wavelength regions. Consequently, a second band-pass filter 128 may be provided that is a broader one that cuts off light in the shorter wavelength regions.

A photodiode 129, which may be a silicon diode, may be incorporated to sense visible light produced by the lamp 112. The photodiode 129 is positioned between the lamp 112 and the band-pass filters 126 and 128, which is prior to the extraction of infrared (IR) resulting from transmission through the band-pass filters. The photodiode 129 monitors the chopper frequency. This is to assure that the timing is known for integration of the dark current.

Referring now to the sampling arm 122 shown in FIG. 3, the light passed by the band-pass filters 126, 128 is input to a piezo-electrically actuated scanning Fabry-Perot etalon 130, which transmits only a portion of the incident light that is within a narrow wavelength range about a desired center wavelength. The width of the wavelength passband of the piezo etalon 130 is determined by the reflectivity of the optical coatings on the etalon 130, while the center wavelength is determined by the thickness of the etalon air gap. The center wavelength can be tuned by changing the voltage applied to the piezoelectric spacer element. The transmitted light is directed through a beam splitter 132 and focusing lenses 134 to the input of a source optical fiber 136 that comprises part of the fiber bundle 50. The source optical fiber 136 in turn routes the light to the sampling region 70, for example, to sample at the interstitial region between the index and middle fingers above the web.

As shown in FIG. 3, a calibration can be performed that employs a calibration arm 124 to calibrate the optical spectrometer 110 after thermal equilibrium is reached. The beam splitter 132 redirects a small portion of the light output by the piezo etalon 130 to the wavelength calibration arm 124. The beam splitter 132 may be constructed from calcium fluoride and may redirect, for example, approximately three percent of the light to the wavelength calibration arm 124. The wavelength calibration arm 124 comprises a Fabry-Perot fixed etalon 138 and a focusing lens 140. The focusing lens 140 focuses the output of the fixed etalon 138 on a wavelength calibration detector 142, which may be an InGaAs detector to detect amplitude, and which may be temperature-controlled by a thermoelectric (Peltier) cooler (TEC) to reduce dark current.

The light emitted from the source optical fiber 136 and impinged on the sampling region 70 is diffuse reflected by the tissue of the person whose blood alcohol concentration is being detected to collection fibers 144. The collection fibers 144 route the received light through focusing lenses 146 to an alcohol signal detector 148, which may be an InGaAs detector, and which may be temperature-controlled by a TEC to reduce dark current. Thus, there are two detectors, the wavelength calibration detector 142 and the alcohol signal detector 148.

As described above, light is delivered onto the tissue by the fiber bundle 50, which may be a bifurcated bundle. In one implementation, the source light is radiated by one fiber 136 having a diameter of 600 microns and is contained in a barrel having a given wall thickness of approximately two hundred microns. Then, the detected light is received through a bundle of other fibers 144 and fed to the alcohol detector 148. There is a small separation between the source fiber 136 and the collection fibers 144. Consequently, only light that penetrates into the tissue to some depth is collected.

The fixed etalon 138 has transmission peaks separated by the desired wavelength sampling interval. The light level at the detector behind the fixed etalon 138 is monitored, and the voltage on the piezoelectric actuator of the piezo etalon 130 is adjusted in order to maximize the light signal. Data is taken at the voltages that correspond to each of the transmission peaks within the desired measurement range. By using the fixed etalon 138 as a reference, data can be taken at a repeatable set of center wavelengths despite hysteresis or other variabilities in the piezoelectric element. Accordingly, the signal from the wavelength calibration detector 142 determines defined sampling points. It also enables internal calibration, because the wavelength calibration detector 142 enables intensity fluctuations in the light source to be monitored.

The disadvantage to the configuration shown in FIG. 3 is that there are two detectors, which increases cost. In accordance with another exemplary implementation, the piezo etalon 130 and fixed etalon 138 may be configured in series. However, a disadvantage to such a series configuration is that there is a reduction of light that contacts the tissue by almost an order of magnitude, because the linewidth decreases from approximately 8 nanometers wide to approximately 1 nanometer wide. Thus, a significant amount of light is lost. In yet another contemplated implementation, the fixed etalon 138 may be eliminated. The disadvantage is that the sampling points may not be set as accurately.

In accordance with other contemplated implementations, the piezo etalon 130 can be replaced with another type of scanning filter, for example:
Liquid crystal (LC) tunable filter (See the CRI product literature, for example). The disadvantage is that LC filters tend to be more expensive than piezo etalons.
Thermo-optically tuned filter (See the Aegis Lightwave product literature, for example).
Microelectromechanical systems (MEMS) based Fabry-Perot etalon. In this case, the air gap of the etalon is tuned by electrostatically or electromagnetically actuating a micro-machined element. The advantages of MEMS-based filters include low cost and small size.

FIG. 4 is an architectural block diagram of the optical spectrometer 110 in accordance with an embodiment of the present invention. The optical portion of the optical spectrometer 110 described above is contained in a housing or box 200. The housing 200 is dust-tight, because a fan (not shown) may be incorporated to cool various elements. Consequently, dust is prevented from blowing into the optics. On the other hand, only a portion 201 of the housing 200 containing the focusing lenses 146 and the alcohol signal detector 148 is lighttight so that no light except light collected from the tissue at the sampling region 70 is detected.

Additionally, the optical spectrometer 110 comprises detector boards 202 and 204 connected to the wavelength calibration detector 142 and the alcohol signal detector 148, respectively, to provide pre-amplification; a driver board 206; and a power supply 208. The driver board 206 comprises a serial port 209 for connection to an analysis system 210 for analyzing the alcohol detection signal and performing dynamic calibration in accordance with an embodiment of the present invention. The detector boards 202 and 204 and driver board 206 may be configured on a PC board that is the same size as the housing 200, so that the PC board forms a lid to the box. A rubber ring may be incorporated around the edge of the PC board. All cabling may be on one side of the PC board, so that the PC board may lift up like a hinge to access to the underside of the PC board, as well as the optics. The power supply 208 may be housed underneath a thick plate to avoid heat transfer and electrical noise.

As shown in FIG. 4, the driver board 206 comprises a lamp driver 212 for the lamp 112, a piezo driver 214 for the piezo etalon 130, and a chopper driver 216 for the chopper motor 120. The photodiode 129 may be combined with a photodiode driver 218 and mounted on the driver board 206. The driver board 206 also comprises temperature controllers 220 and 222 for cooling the image detectors 142 and 148, respectively, and a processor 224 and a driver 226 for the serial port 209 coupled to a computer 228 for analysis.

Additionally, as shown in FIG. 4, the optical spectrometer 110 in accordance with one implementation comprises a contact switch 240 and indicator LED 230 to assure that there has been contact made with the tissue at the sampling region 70, because the fiber bundle 50 may not be completely covered by the tissue. The contact switch 240 may be positioned directly above the fiber bundle 50 so that only when there is complete contact with the tissue is the electrical signal sent that indicates data may be acquired. The driver board 206 comprises a driver 232 for the contact switch 240 and indicator LED 230.

In addition, as shown in FIG. 4, a diffuse reflectance surface, such as a Spectralon disc 234, may be employed for white light calibration, for example, a few times a day, or before each sample is measured. Consequently, while the fixed sampling positions are determined by the fixed etalon 138 depending on the wavelength, it may also be useful to provide a calibration for the signal obtained from the tissue, which is a diffuse reflectance signal. The calibration accounts for the shape to the light that finally reaches the tissue due to the band-pass filters 126, 128 and piezo etalon 130 and other optics. Therefore, instead of having a flat source of light impinging on the tissue, there is a shape to the source light that is determined using a diffuse reflectance surface such as the Spectralon disc 234.

The Spectralon disc 234 may serve as a reference channel inside the spectrometer. That is, by shining light on a known reference standard, such as the Spectralon disc 234, a spectral signature for a known wavelength can be detected. The band-pass filter (e.g., the piezo etalon 130) can then be adjusted so that the spectral signature is at the specified wavelength.

In an exemplary embodiment according to the present invention, the reference surface (for example, Spectralon) is part of the hand cradle 80. For instance, it may form a door or shutter 234 to occlude the probe head 40 or fiber optic bundle 50 and reflect the signal delivered to the fiber optic bundle 50. In another embodiment, the surface may be part of an automatic door 234 that rolls up and down (like a garage door), or swings in or out of the way, or opens and shuts (like a camera shutter) to occlude the probe head 40 or fiber optic bundle 50. The diffuse reflectance surface (for example, Spectralon) would be part of the door or shutter (e.g., mounted to the door), and face the probe head to allow for a reference check, such as for calibrating for dark current.

In operation, the reference door 234 may normally be shut or closed, thus occluding the probe head 40. The system may want to perform a reference check before each actual reading of a human tissue sample. Thus, when the cradle senses that an actual reading is about to take place, the system can initiate a reference check against the Spectralon surface before moving the reference surface 234 out of the way (e.g., “rolling up the garage door”) to permit the probe head 40 to do an actual spectroscopic sampling of the human tissue.

The calibration employing the Spectralon disc 234 may actually be employed for two purposes. First, the calibration may be performed while slowly scanning through the operating spectrum to locate the voltage values for the piezo etalon 130 that correspond to the desired sampling positions. Second, the Spectralon calibration is performed a few times a day, for example, to obtain a blank measurement to the background, that is, what light is detected by radiating the Spectralon disc 234. Then, when actual tissue is sampled, the resulting data is divided by the calibration data to yield the alcohol concentration data.

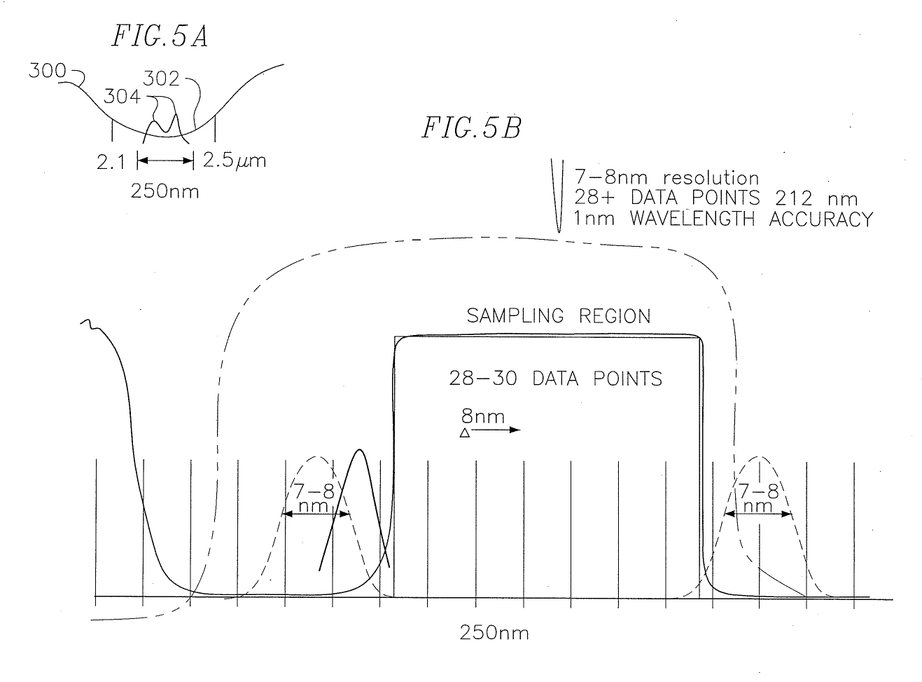

In operation, the coatings for the band-pass filters 126, 128 are intended to pass a particular wavelength range associated with detection of blood alcohol concentration, for example. This particular wavelength range is in the 2.1-2.5 micron range, as shown in FIG. 5A. The particular wavelength range of interest is one in which water is generally highly absorptive, as indicated by the absorption curve 300. However, within the range of interest, a dip 302 occurs in the water absorption spectrum, and coincident with the dip, there are several peaks 304 associated with alcohol, that have a higher absorption than water in that narrow region. This region is selected for analysis, because a contribution due to the presence of alcohol can be detected. This region is in the approximate wavelength region of approximately 2.1 to 2.5 microns, and, more particularly, can be in the range of approximately 2150 to 2400 nanometers, and is the actual wavelength scan region for light supplied by the piezo etalon 130.

The alcohol concentration sampling window region corresponding to the dip 304 only occupies approximately 250 nanometers of the 2.1-2.5 micron range (which is in the high near-infrared spectral region), as shown in FIG. 5A. 250 nanometers is a relatively small wavelength region, especially when considering the wavelength range over which large spectrometers such as a Fourier Transform InfraRed (FTIR) based on a Michelson Interferometer or a standard dispersive spectrometer employing a grating operate. However, Michelson Interferometers are expensive and bulky while a dispersive spectrometer that uses a diffraction grating to disperse the light across an InGaAs array detector is also expensive due to the cost of the array detector. In contrast, as described above, one implementation of the optical spectrometer 110 shown in FIG. 3 includes two detectors 142, 148, or in another implementation uses only one detector.

Accordingly, as shown in FIG. 5B, the approximately 250 nanometer window in the 2.1-2.5 micron range region is scanned. Specifically, by applying different voltages across a piezo comprising the piezo etalon 130, the light incident on the piezo etalon is stepped through the wavelengths of light within the window. In accordance with an exemplary implementation, the piezo etalon 130 is actually stepped with 7 to 8 nanometer wavelength resolution. Additionally, the fixed etalon 138 has a resolution of one nanometer spaced every 7 to 8 nanometers. Consequently, the piezo etalon 130 in combination with the fixed etalon 138 produce approximately 28 data points across a 212 nanometer region corresponding to the sampling window, and having a linewidth of one nanometer with one nanometer wavelength accuracy. The data points are at known wavelengths.

The optical spectrometer 110 actually collects diffuse reflectance, so when light impinges on tissue, the tissue is very highly scattering. The light undergoes multiple scattering absorption steps, such that the optical properties of the tissue are sampled, and the diffuse reflectance received from the issue is then collected by the collection fibers 144. The alcohol contribution is approximately 0.3 percent of the tissue diffuse reflectance. Consequently, a signal-to-noise of approximately 100 is needed in order to discern the alcohol concentration signal.

A voltage may be applied to the piezo etalon 130 by the piezo-electric driver 214, which includes any needed correction for the creep and hysteresis of the piezo. If a voltage is applied to scan very slowly across the alcohol concentration scanning window region, the scan time is relatively long. The scan may be performed in approximately five seconds or less to obtain the data points for analysis of the blood alcohol concentration. Accordingly, one implementation determines the voltage values that correspond to each one of the wavelengths employed for alcohol concentration analysis, which requires calibration since the operation of the piezo comprising the piezo etalon 130 is subject to variation for reasons such as thermal drift.

In order to perform the calibration, a diffuse reflectance surface, such as the Spectralon disc 234, is employed. The light impinged on the Spectralon disc 234 during calibration produces a sufficient diffuse reflectance signal for calibration.

Accordingly, the piezo etalon 130 is scanned slowly across the alcohol concentration scanning window employing the Spectralon disc 234 for calibration, and the voltages applied to the piezo are acquired at which peak diffuse reflectance from the Spectralon disc 234 are detected corresponding to the sampling points. The values of the voltages are stored in a lookup table by the processor 208. The voltages stored in the lookup table are then applied to the piezo etalon 130 to produce the wavelengths corresponding to the sampling points in the alcohol concentration scanning window region, so that when a person inserts his or her hand, the piezo etalon can quickly jump to each one of the wavelength sampling positions. The scan speed is fast, because the voltages for the piezo comprising the piezo etalon 130 may be applied to jump from one sampling wavelength to the next. In another embodiment, they may be reset to zero before the voltage is applied so that the piezo etalon jumps to the next sampling wavelength if needed to correct for creep and hysteresis.

While diffuse reflectance occurs relatively quickly, there is a finite amount of time required for the piezo etalon 130 to jump to a wavelength sampling position and then settle. The settling time is relatively short, about a millisecond or less in an exemplary implementation. However, in accordance with one implementation, each integration involves both a signal integration and a dark current integration. For this implementation, an additional period of approximately 30 milliseconds is also provided between each wavelength sampling point while scanning to provide sufficient time during which there is no signal on the alcohol detector 148 to enable integration of the dark current to be performed.

In accordance with another implementation, multiple scans may be preformed. That is, one scan across the alcohol concentration sampling window region is performed, then one or more additional scans are performed. Each one of these spectra may be used to obtain an average for multivariate calibration analysis while maintaining information respecting the third and fourth moments to aid the analysis.

The data for the resulting spectra may be converted to information regarding a chemical substance or analyte present in the blood by way of multivariate calibration techniques (e.g., principal component regression (PLS), classical least squares (CLS), and partial least squares (PCR) regression models). Multivariate calibration is employed, because the alcohol concentration detection signal is on the order of 0.3 percent within the spectrum that results by scanning across the alcohol concentration scanning window region, which is too small to perform peak analysis. Consequently, multiple different spectra are acquired for which the alcohol concentrations are known, for example, by employing a blood draw or by employing an evidentiary breathalyzer device to provide reference values. These multiple different spectra are obtained from different people or from the same person at multiple alcohol concentration levels, for many people/persons. As a result, reference spectra are stored, for which the corresponding alcohol concentrations are known.

Then, to determine the blood alcohol concentration, the reference spectra are employed to generate a regression vector or B vector. The concentration of interest is the spectrum, or average of the detected spectra, obtained by scanning across the alcohol concentration scanning window dotted (i.e., multiplied) with the B vector. To determine the unknown blood alcohol concentration, the B vector is multiplied by the spectrum or average of spectra that has been detected, to yield a blood alcohol concentration. From this perspective, the multivariate calibration step can be regarded as the calculation of a regression vector, whose length is the amount of net signal when the value of the property of interest (e.g., blood alcohol concentration) is equal to a known blood alcohol concentration. The determination step can be interpreted as projecting the detected spectrum onto the direction of the net regression vector. The length of the detected spectrum divided by the length of the net regression vector is the value of the property of interest, namely, the detected blood alcohol concentration.

The multivariate calibration technique may be employed to perform quantitative analysis respecting the alcohol spectrum detected by scanning across the alcohol concentration scanning window to yield a blood alcohol concentration measurement, for example, 0.06% blood alcohol concentration. In another embodiment, rather than performing a quantitative measurement, a classification may be provided, for example, zero blood alcohol concentration, less than 0.07%, or greater than 0.07%.

One implementation acquires both spectral information indicative of blood alcohol concentration, as well as a biometric verification. The biometric verification is employed to confirm the identity of the person whose blood alcohol concentration is detected.

Another implementation acquires both spectroscopic information related to blood alcohol concentration and information related to other physiological parameters such as tissue oxygenation or lactic acid concentration. The measurement of these alternative physiological parameters is used to confirm that a valid biological sample is presented to the instrument for measurement. In addition, the measurement of these additional parameters can be used to correct for variability in the optical transmission of tissue and, hence, can improve the accuracy and/or precision of the blood alcohol measurement.

In FIG. 6, in accordance with an embodiment of the present invention, dynamic calibration is additionally provided for the drive voltages applied to the piezo-electrically actuated scanning Fabry-Perot etalon 130, for example, to adjust the drive voltages to compensate for drift due to changes in temperature. The particular implementation of optical spectrometer 110 incorporates a Fabry-Perot filtering element 130 that comprises two carefully mirrored surfaces. The mirrors are determined for the wavelength of interest, narrowly spaced, and they are moved on an optical axis by a piezoelectric element 131, so that by changing the mirror spacing, one can tune the frequency of light that can pass through the assembly.

The light that is filtered by the piezo etalon 130 has a narrow Gaussian-shaped peak, which may be approximately 20 nanometers wide. By applying a varying voltage to the piezoelectric element, one can scan that peak across the optical region of interest.

The piezo etalon 130 is an open loop device. That is, when a drive voltage is applied, the piezoelectric element simply moves the mirrors. There is no measurement module or measurement technique that guarantees that the mirrors in fact move to provide a particular air gap between them (that is, calibrates the drive voltages with the resulting air gaps and hence, passbands of the piezo etalon 130). In one implementation, the optical components consist of fused quartz, and the piezoelectric element is a ceramic.

The piezo etalon 130 is susceptible to thermal drift due to temperature changes that can affect precision of the optical spectrometer 110. Care should be exercised to construct the optical spectrometer 110 to minimize the problem of thermal instability. For example, in an exemplary embodiment, a high power light bulb 112 is present. Care should be exercised so that unwanted light is not absorbed into the metal of the housing creating a source of heat that is transferred to the piezo etalon 130. Further examples of heat sources include heat sinks for the Peltier coolers cooling the two detector elements and other electronics as well as the chopper motor 120 that rotates the chopper wheel 118. All of these heat sources should be placed as far away from the piezo etalon 130 as possible and thermally isolated to assure that heat does not flow through the optical assembly and produce thermal excitation of the piezo etalon. Otherwise, the risk is that the optical spectrometer will drift out of usable range quite quickly. By removing these sources of heat, the optical spectrometer has a much smaller temperature excursion during the day and reaches its own internal thermal equilibrium sooner after being placed in operation.

Notwithstanding these design precautions, the optical spectrometer 110 may still be subjected to sudden changes in ambient temperature, such as when moving the device from one environment (for instance, inside a vehicle) to another (for example, inside a building). In addition, the fixed etalon 138 is affected by temperature changes, which can adversely affect the calibration.

Consequently, to calibrate during warm-up of the optical spectrometer and to otherwise calibrate to compensate for thermal drift due to changes in temperature, dynamic calibration is provided in accordance with an embodiment of the present invention. Dynamic calibration in accordance with an embodiment entails widening the scanning range of the optical spectrometer 110 a small amount to enable the spectrometer to analyze the wavelengths of the light passed by the band-pass filters 126, 128. In the optical spectrometer 110, the light is processed by the optical band-pass filters 126, 128 that only pass near infrared wavelengths from approximately 2180 nanometers to approximately 2400 nanometers.

By widening the scanning range of the piezo etalon 130, the optical spectrometer 110 can measure the amplitude of the wavelengths passed by the band-pass filters 126, 128 using a reference sample, such as Spectralon. Then, by performing computer-based calculations based on those measurements, the piezo etalon 130 can be aligned using the passband of the band-pass filters 126, 128, and the drive voltages for the piezoelectric element of the piezo etalon 130 can be adjusted to scan across the appropriate wavelength range within the passband. The drive voltages to the piezoelectric element can be continually updated to assure that the piezo etalon 130 always scans the correct wavelength range. As a result, the drive-voltage-to-wavelength correlation can continually be updated so that the computer-based calculations can report a data value at the correct frequency, as compared to only reporting the drive voltage, which in a first order sense is uncorrelated with the frequency due to factors such as thermal drift.

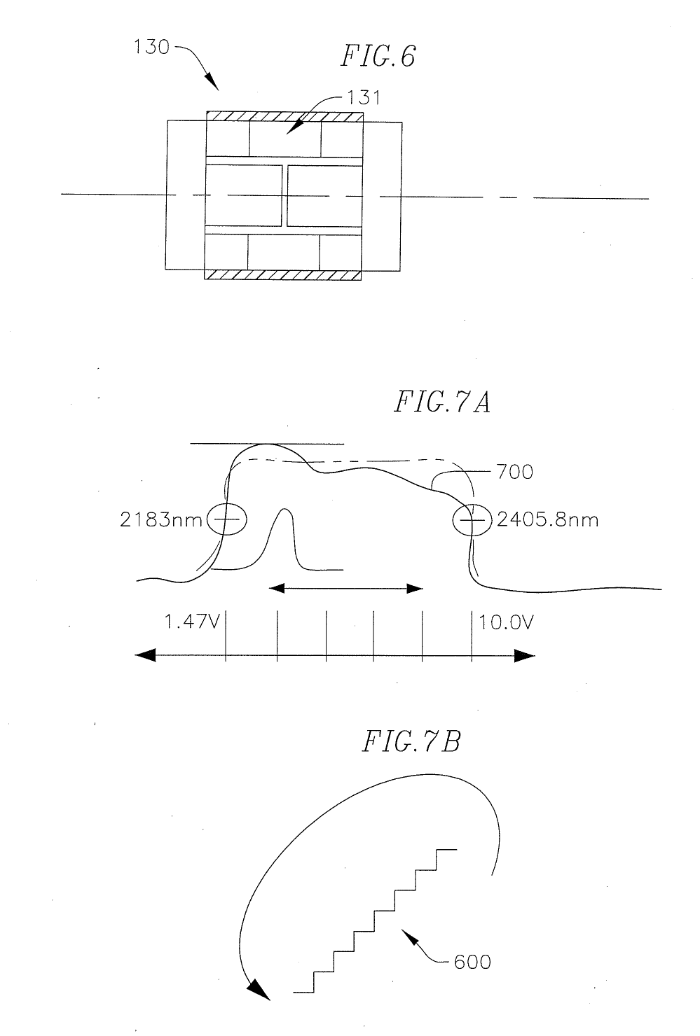

Considered in more detail, FIG. 6 shows a cross-section of an exemplary piezo etalon 130 wherein the light path is along the horizontal axis. In this example, the piezoelectric element 131 is cemented between two quartz rings, and the outside ends of the quartz rings are afterwards lapped and contact bonded to the end windows. The piezoelectric element 131 controls the spacing of an air gap and multiple coating windows on either side of this gap form the tunable transmissive element that comprises the Fabry-Perot interferometer. By changing the spacing, the peak shown in FIG. 7A moves over a range. There are multiple frequencies that this peak can satisfy, but only one in the wavelength interval of interest, so a band-pass filter is incorporated to constrain the system to the desired peak. The band-pass filter can be incorporated, for example, between the light source and the input to the piezo etalon 130, but it could also be anywhere in the optical path of the optical spectrometer. This band-pass filter can be quite well defined, as shown in FIG. 7A.

In accordance with the dynamic calibration method of the present invention, the scanning range is extended so the piezo etalon 130 scans across the passband shown in FIG. 7A, which has a characteristic response 700 to the wavelengths that are input to the band-pass filter, such that the response curve has a shape such as shown. Accordingly, using a reference sample such as Spectralon, the amplitude of the wavelength response can be measured, and then the two 50% responses (half-magnitude points) at the ends of the passband can be determined algorithmically.

Now, since the characteristic response of the band-pass filters 126, 128 is known, the 50% responses correspond to exact wavelengths. Though this may vary between different sets of band-pass filters, an example set may have 50% responses corresponding to 2183 nanometers and 2405.8 nanometers, respectively. For this example set, based on aligning the piezo etalon 130 to the band-pass filters 126, 128, at the given point in time, the drive voltage for the piezoelectric element that results in 50% responses at 2183 and 2405.8 nanometers can be determined, for example, 1.477 volts and 10.0 volts, respectively. That determines a drive voltage range over the example wavelength range of 2183 nanometers to 2405.8 nanometers, and dividing the difference between those drive voltages by the number of sample points yields the drive voltage increments for the staircase voltage function 600 shown in FIG. 7B to produce the sample points in absolute volts for performing measurements. There is hysteresis in the piezoelectric element, but since the scan is from one point to the other in a staircase and then returns to the beginning point, the hysteresis event is outside of the calibration scan.

A calibration operation can be performed at normal intervals, for instance, at the end of every patient operation. It can also be performed in response to environmental stimuli, such as when there is a two-tenths of a degree change in the ambient temperature within the housing. In addition, it can be performed at certain time intervals, though these may need to be more frequent when the optical spectrometer 110 is first turned on, before the optical spectrometer reaches thermal equilibrium. Once the optical spectrometer approaches thermal equilibrium, these time periods can be stretched out.

While the foregoing description has been with reference to particular embodiments and contemplated alternative embodiments of the present invention, it will be appreciated by those skilled in the art that changes in these embodiments may be made without departing from the principles and spirit of the invention. For example, dynamic calibration in accordance with an embodiment of the present invention is applicable to any system with a band-pass device and a device that scans, such that the scan window can be widened, and the edges of the band-pass device can be used to calibrate out drifts in equipment. Accordingly, the scope of the present invention can only be ascertained with reference to the appended claims.

What is claimed is:

1. A method of dynamic calibration of an optical spectrometer comprising a spectroscopic detector for analyzing a spectroscopic sample of an analyte obtained through directing a tunable source of electromagnetic radiation at the tissue of a person through a probe, the method comprising:
directing a portion of the electromagnetic radiation to a wavelength calibration detector;
measuring the electromagnetic radiation at the wavelength calibration detector; and
tuning the source of electromagnetic radiation using the wavelength calibration detector measurements to correspond to a desired wavelength of the electromagnetic radiation.

2. The method of claim 1, wherein the optical spectrometer further comprises a diffuse reflectance surface for calibrating the spectroscopic detector, the method further comprising directing the source of electromagnetic radiation to the diffuse reflectance surface to provide a spectroscopic baseline sample for the spectroscopic detector.

3. The method of claim 2, wherein the optical spectrometer further comprises a shutter or door for occluding the probe and the shutter or door comprises the diffuse reflectance surface, the method further comprising:
occluding the probe with the shutter or door; and
acquiring the spectroscopic baseline sample.

4. The method of claim 3, wherein the shutter or door is further for automatically and temporarily occluding the probe, the method further comprising automatically and temporarily occluding the probe for obtaining the spectroscopic baseline sample at a time proximal to obtaining the spectroscopic sample of the person.

5. The method of claim 2, wherein the optical spectrometer further comprises a band-pass filter for filtering the electromagnetic radiation to a desired wavelength range, the method further comprising:
acquiring a plurality of spectroscopic baseline samples of the diffuse reflectance surface; and
tuning the source of electromagnetic radiation using the baseline samples to deliver the electromagnetic radiation at a plurality of desired wavelengths within the desired wavelength range.

6. A dynamically calibrating optical spectrometer for acquiring and analyzing a spectroscopic sample of an analyte from a sampling region of the tissue of a person, the optical spectrometer comprising:
a tunable source of electromagnetic radiation;
a probe for delivering the electromagnetic radiation to the tissue at the sampling region and obtaining a diffuse reflectance signal from the tissue at the sampling region;
a spectroscopic detector for analyzing the diffuse reflectance signal for presence of the analyte; and
a wavelength calibration detector for calibrating the tunable source of electromagnetic radiation to a desired wavelength.

7. The optical spectrometer of claim 6, further comprising a diffuse reflectance surface for providing a spectroscopic baseline sample for calibrating the spectroscopic detector.

8. The optical spectrometer of claim 7, wherein the diffuse reflectance surface is configured to temporarily occlude the probe for acquiring the spectroscopic baseline sample.

9. The optical spectrometer of claim 8, wherein the diffuse reflectance surface is configured to automatically occlude the probe, for acquiring the spectroscopic baseline sample at a time proximal to acquiring the spectroscopic sample of the person.

10. The optical spectrometer of claim 8, further comprising a door or shutter comprising the diffuse reflectance surface.

11. The optical spectrometer of claim 7, wherein the diffuse reflectance surface comprises Spectralon.

12. The optical spectrometer of claim 7, further comprising a band-pass filter for filtering the electromagnetic radiation to a desired wavelength range, wherein the optical spectrometer is further for using a plurality of spectroscopic baseline samples of the diffuse reflectance surface to tune the source of electromagnetic radiation to deliver the electromagnetic radiation at a plurality of desired wavelengths within the desired wavelength range.

13. The optical spectrometer of claim 6, wherein the analyte is alcohol.

14. The optical spectrometer of claim 6, further comprising a biometric sensor for verifying the identity of the person.

15. The optical spectrometer of claim 14, wherein the biometric sensor is a fingerprint scanner.

16. The optical spectrometer of claim 6, further comprising a calibration arm for directing a portion of the electromagnetic radiation from the tunable source to the wavelength calibration detector.

17. The optical spectrometer of claim 16, wherein the calibration arm comprises a fixed etalon.

18. The optical spectrometer of claim 17, further comprising an adjustable etalon for tuning the source of the electromagnetic radiation to the desired wavelength.

19. A dynamically calibrating optical spectrometer for acquiring and analyzing a spectroscopic sample of an analyte from a sampling region of the tissue of a person, the optical spectrometer comprising:
a tunable source of electromagnetic radiation;
a probe for delivering the electromagnetic radiation to the tissue at the sampling region and obtaining a diffuse reflectance signal from the tissue at the sampling region;
a spectroscopic detector for analyzing the diffuse reflectance signal for presence of the analyte; and
a diffuse reflectance surface for providing a spectroscopic baseline sample for calibrating the spectroscopic detector.

20. The optical spectrometer of claim 19, wherein the diffuse reflectance surface is configured to automatically occlude the probe, for acquiring the spectroscopic baseline sample at a time proximal to acquiring the spectroscopic sample of the person.
